(19) United States
(12) Patent Application Publication
(10) Pub. No.: US 2017/0019906 A1
(43) Pub. Date: Jan. 19, 2017

(54) Method for Mitigating Interference Between Two or More Wide Body Area Networks

(71) Applicant: KABUSHIKI KAISHA TOSHIBA, Tokyo (JP)

(72) Inventor: Siva Kupanna SUBRAMANI, Bristol (GB)

(21) Appl. No.: 15/124,713

(22) PCT Filed: Jul. 7, 2014
(86) PCT No.: PCT/GB2014/052064

§ 371 Date: Sep. 9, 2016

(51) Int. Cl. H04W 72/08; H04W 40/24; A61B 5/00; H04B 13/00

(52) U.S. Cl. CPC H04W 72/082; H04B 13/005; H04W 40/244; A61B 5/0024; A61B 5/7217; A61B 2560/0266

(57) ABSTRACT

A method of mitigating interference between two or more wide body area networks WBANs, the method comprising: detecting, at a first WBAN, control channel beacons transmitted from one or more other WBANs, each control channel beacon specifying a data channel on which the respective WBAN is operating; determining, based on the detected control channel beacons, whether one of the other WBANs is operating on the same data channel as the first WBAN and if so, adjusting one or more communication parameters of the first WBAN or requesting said one of the other WBANS to adjust its own communication parameters in order to mitigate interference between the first WBAN and said one of the other WBANs.

### FIELD

Embodiments described herein relate to systems and methods for mitigating interference between wireless body area networks WBANs.

### BACKGROUND

A wireless body area network (WBAN) is a network of sensor nodes designed to be carried by a person and used for monitoring, logging and transmitting vital healthcare signals from that person. FIG. 1 shows an example of two WBANs 101a, 101b worn by respective individuals. Each WBAN consists of multiple sensor nodes that transmit to a hub/coordinator node. The first WBAN 101a includes sensor nodes 103a that transmit at a constant bit rate (high data rate) to the hub. The second WBAN 101b includes sensor nodes 103a that transmit at a constant bit rate (high data rate) to the hub and further comprises other sensor nodes 103b that transmit at an intermittent bit rate (low data rate) to the hub. In general, a WBAN may comprise one or other, or a combination of these different types of sensors nodes. The sensor nodes tend to be extremely low powered with transmission ranges of only a few meters. For simplicity, each individual can be considered to comprise a WBAN.

A challenge faced when working with WBANs is that they are randomly distributed and move about with people to whom they are attached. A WBAN is likely to interfere with other WBANs when those networks come within each others' transmission range. Such interference may potentially result in the loss of life-critical information, and is likely to be of particular concern in dense network scenarios, such as crowded locations, hospitals, etc. Conventional methods for managing interference between WBANs include a ‘carrier sensing’ approach, in which the hub assesses the availability of the medium through carrier sensing and performs a clear channel assessment (CCA) by comparing the detected energy level against a threshold.

### BRIEF DESCRIPTION OF FIGURES

Embodiments of the invention will now be described by way of example with reference to the accompanying drawings in which:

FIG. 1 shows an example of two wireless body area networks WBANs worn by respective individuals;

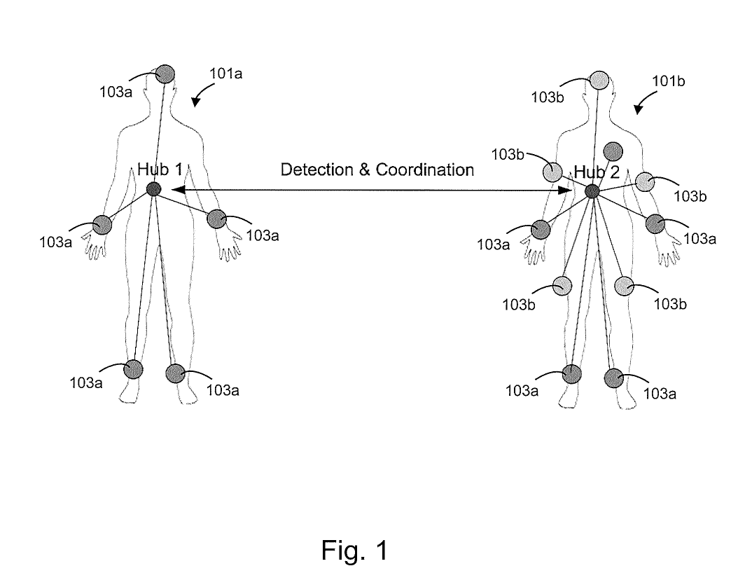

FIG. 2 shows the format of a control channel beacon broadcast by a WBAN in an embodiment;

FIG. 3 shows the timing of broadcasts of control channel beacon frames from a WBAN in an embodiment;

FIG. 4 shows the format of a data channel superframe broadcast by a WBAN in an embodiment;

FIG. 5 shows a flow chart of steps in a method of mitigating interference between two or more WBANs according to an embodiment;

FIG. 6 shows an example of how a hub may use one or more transceivers to scan for control channel information and data channel information in embodiments described herein;

FIG. 7 shows an example of control channel beacons being broadcast at intervals by different WBANs, on different control channels;

FIG. 8 shows an example of how a distance between hubs of respective WBANs may determine whether or not a first one of those hubs receives control channel information from the other one of the hubs;

FIG. 9 shows a flow chart of steps in a method of mitigating interference between two or more WBANs according to an embodiment;

FIG. 10 shows a flow chart of steps in a method of mitigating interference between two or more WBANs according to an embodiment;

FIG. 11 shows an example of how a hub in a first WBAN may extend the duration of its control/management period in order to overlap with that of a second WBAN that is determined to be operating on the same data channel as the first WBAN;

FIG. 12 shows a flow chart of steps in a method of mitigating interference between two or more WBANs according to an embodiment;

FIG. 13 shows a flow chart of steps in a method of mitigating interference between two or more WBANs according to an embodiment;

FIG. 14 shows an example of how a peripheral sensor node in a first WBAN may encounter interference from a second, neighbouring WBAN, where the hub of the first WBAN itself lies out of range of the second WBAN;

FIG. 15 shows an example of how the peripheral node of FIG. 14 may notify the hub of the presence of the second WBAN, during the control/management period of the hub; and

FIG. 16 shows a computing device according to an embodiment.

### DETAILED DESCRIPTION

According to a first embodiment, there is provided a method of mitigating interference between two or more wide body area networks WBANs, the method comprising:

detecting, at a first WBAN, control channel beacons transmitted from one or more other WBANs, each control channel beacon specifying a data channel on which the respective WBAN is operating;

determining, based on the detected control channel beacons, whether one of the other WBANs is operating on the same data channel as the first WBAN and if so, adjusting one or more communication parameters of the first WBAN or requesting said one of the other WBANS to adjust its own communication parameters in order to mitigate interference between the first WBAN and said one of the other WBANs.

In some embodiments, the first WBAN adjusts its one or more communication parameters by switching to operate on a different data channel.

In some embodiments, the first WBAN adjusts its one or more communication parameters by adjusting the timing of an active period in the data channel on which it is operating.

In some embodiments, the first WBAN maintains a record of WBAN activity in the vicinity. The record may include a list of hubs from which control channel beacons have been detected and the data channels on which those hubs are operating. The record may be updated on receipt of subsequent control channel beacons at the first WBAN. In some embodiments, the record of WBAN activity is used to generate statistics reflecting the duration for which each particular hub is active in a data channel, the statistics being updated on receipt of subsequent control channel beacons at the first WBAN. The statistics may include the mean duration for which each hub is active in a data channel and/or the standard deviation in the duration for which each hub is active in a data channel. In some embodiments, the record is stored in the form of a table.

In some embodiments, the first WBAN notifies said one of the other WBANS of the possibility of interference between the first WBAN and said one of the other WBANs by:

determining a control/management period of said one of the other WBANs;

extending a control/management period of the first WBAN so as to coincide with the control/management period of said one of the other WBANs;

constructing a message for sending from the first WBAN to said one of the other WBANS; and

sending the message to said one of the other WBANs during the control / management period.

In some embodiments, the first WBAN determines if the interference can be avoided by switching to operate a different data channel and if not, the first WBAN notifies said one of the other WBANS of the possibility of interference between the first WBAN and said one of the other WBANs by:

determining a control/management period of said one of the other WBANs;

extending a control/management period of the first WBAN so as to coincide with the control/management period of said one of the other WBANs;

constructing a message for sending from the first WBAN to said one of the other WBANS; and

sending the message to said one of the other WBANs during the control/management period.

In some embodiments, the first WBAN scans a plurality of available data channels in order to determine whether the interference can be avoided by switching to a different data channel.

In some embodiments, the step of scanning the plurality of channels is carried out subject to the probability of finding a free data channel being above a threshold.

In some embodiments, the first WBAN uses the message to request the said one of the other WBANS to adjust its communication parameters in order to mitigate interference between the first WBAN and said one of the other WBANs.

In some embodiments, the message is unicast addressed to said one of the other WBANS.

In some embodiments, the first WBAN comprises one or more peripheral nodes and a hub,

wherein the one or more control channel beacons are detected at the peripheral node(s) and the information contained in the control channel beacons is relayed from the peripheral nodes to the hub, the hub determining whether to adjust one or more communication parameters of the WBAN on the basis of the received information.

According to a second embodiment, there is provided a computer device for use as a node in a first wireless body area network WBAN, the computer device being configured to receive information contained in control channel beacons transmitted from one or more other WBANs;

the computer device comprising:
a data channel identification module for identifying, based on the received information, data channels on which the other WBANs are operating; and
an interference mitigating module configured to determine whether one of the other WBANs is operating on the same data channel as the first WBAN and if so, to adjust one or more communication parameters of the first WBAN or initiate a request for said one of the other WBANS to adjust its own communication parameters in order to mitigate interference between the first WBAN and said one of the other WBANs.

According to a third embodiment, there is provided a non-transitory computer readable storage medium comprising computer executable instructions that when executed by a computer will cause the computer to carry out the method of the first embodiment.

Embodiments described herein use control channel and/or data channel beacon information to detect other WBANs in the vicinity. The control channel and/or data channel beacon information can be used to detect a WBAN or multiple WBANs that are using the same frequency channel, and which could therefore interfere when within transmission range. Embodiments are able to use the control channel(s) for inter-hub detection and communication, for example.

In some embodiments, instead of reacting after suffering packet losses, a pre-emptive communication is initiated that is unicast addressed directly to a detected WBAN. The pre-emptive communication may be initiated in the specific active period of the detected WBAN.

Where the networks are homogeneous networks i.e. the network devices of same type are considered, the control channel and/or data channel beacon information may be obtained through listening to the beacons and decoding the frame packets using same RAT and frame structures.

For simplicity, it will be assumed that the frequency range assigned to WBANs in embodiments described herein is in the universal ISM 2.4-2.485 GHz band. However, it will be understood that it is not essential for the WBANs to operate in this frequency range; embodiments are equally compatible with US MBANs (2.36-2.4 GHz) and European MBANs (2.485-2.5 GHz), as well as other frequency ranges.

In the following, it will further be assumed that the frequency range is split in to 40 channels, each channel having a width of 2 MHz, where the central frequency f_{c} of each channel is given by:

f_{c}=2402+2*n MHz, where n=1 to 40.

The 40 channels comprise 3 control channels and 37 data channels. The control channels are used to transmit control messages in the form of control channel beacons from the hub of the WBAN. The data channels can be used to transmit both data and control messages. The data channels may be used by the peripheral sensor nodes of the WBAN to transmit to the hub (i.e. uplink transmission) and/or vice versa.

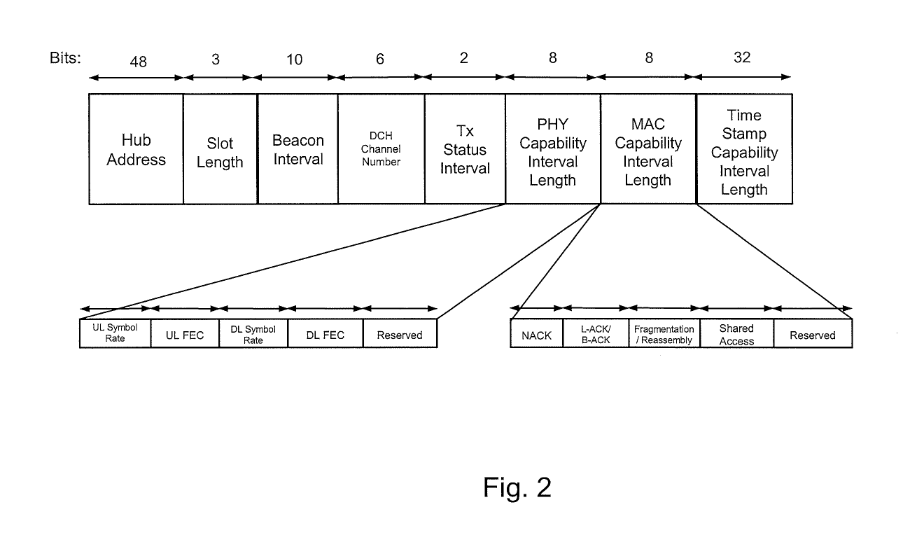

An example of a control channel beacon frame is shown in FIG. 2 and consists of the following 8 fields:
Hub Address
Slot Length
Beacon Interval
Channel Number
Transmission Status (Tx Status)
PHY Capability
MAC Capability
Time Stamp

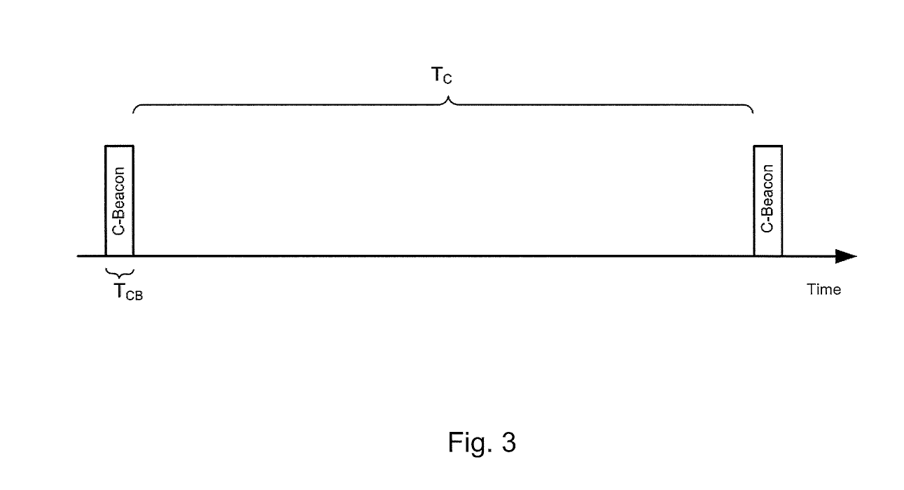

As shown in FIG. 3, the hub will transmit a control channel beacon frame (C-Beacon) on a preselected one of the control channels, every T_{C} seconds. The control channel beacon itself has a duration of T_{CB} seconds.

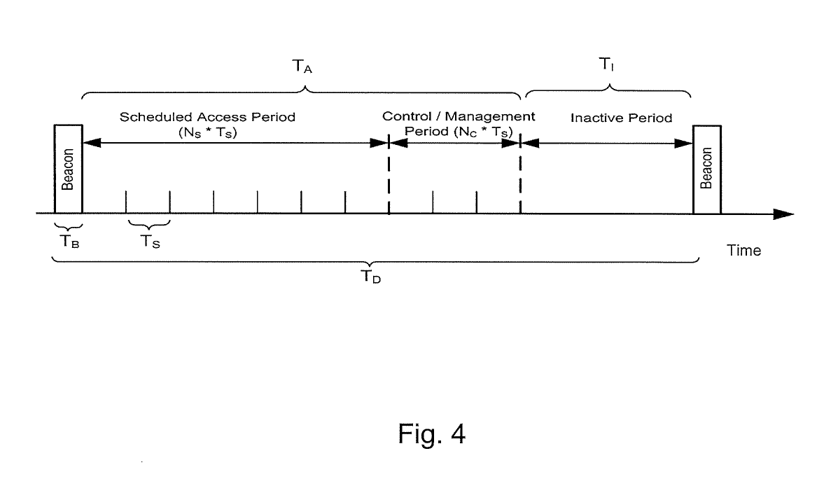

Within each data channel, the time axis is divided into periodic frames referred to as “superframes” of equal length. FIG. 4 shows an example format of such a superframe. The superframe is composed of slots of equal length T_{S} and numbered from 0, 1, . . . , s, where S≦255. Each superframe consists of three parts: a data beacon slot of period T_{B}, an active period T_{A}, and an inactive period T_{I}. During the active period, the hub is operable to either transmit or receive data to/from the peripheral sensor nodes of the WBAN. The active period T_{A} itself comprises a scheduled access period of length N_{S}*T_{S} and a control management period of length N_{C}*T_{S}, where N_{S} and N_{C} are the number of slots in the scheduled access period and control management period, respectively. The peripheral nodes send their data to hub during the scheduled access period, in scheduled time slots. The Control and Management period is devoted for unscheduled, control and management signalling (as distinct from the exchange of sensor data that takes place between the peripheral sensor nodes and the hub during the scheduled access period, for example). When in the inactive period, the WBAN coordinator enters “sleep mode” to reduce energy consumption. The hub does not communicate with the nodes (i.e. does not transmit or receive from the nodes) during the inactive period.

Each superframe is bounded by the data channel beacons, hence the beacon interval BI represents the superframe period T_{D}; for tractable analysis BI=T_{D}=2^{BO} where BO is beacon order. The WBAN will select the beacon order and hence the superframe period depending on the application sensor(s) attached. Therefore, it is possible that different WBANs may have superframes of different lengths.

As distributed WBANs move around, they are likely to interfere with other WBANs as they enter one another's interference range (R_{I}). When within transmission range (R_{T}), the hubs are able to communicate each other and listen to each other's beacons.

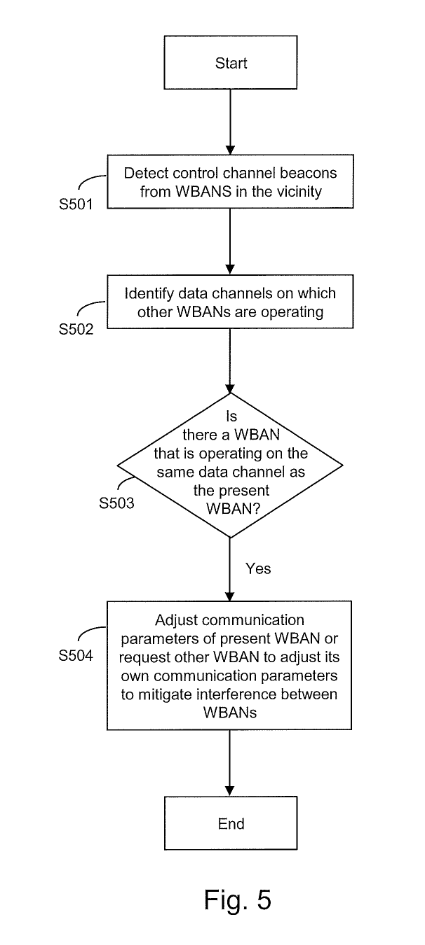

FIG. 5 shows an example of a method in which a WBAN mitigates interference between itself and another WBAN, according to an embodiment. Commencing with step S501, the WBAN scans the control channels to detect control channel beacons transmitted from the hubs of neighbouring WBANs in the vicinity, and which are within range.

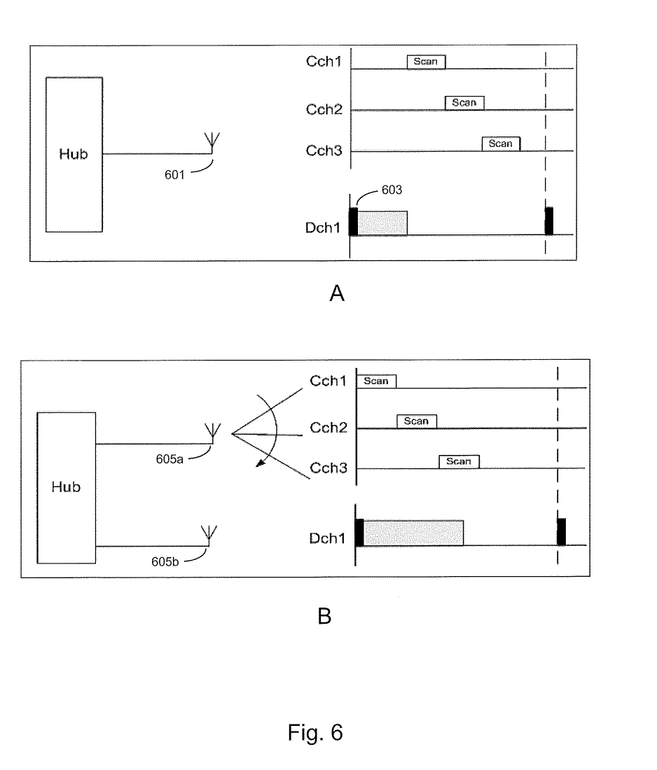

A hub may scan the control channels using a single transceiver 601 (RF chain), as shown in FIG. 6A. Here, the hub may alternate between scanning the control channels and data channel(s). For example, as shown in FIG. 6A, the hub may transmit a data channel beacon 603 in the data channel Dch1 and then wait to receive data packets sent from the peripheral sensors of the WBAN on that channel. After a certain (predetermined) amount of time, the hub may switch to sequential scanning of the control channels Cch1, Cch2 and Cch3 in order to detect control channel beacons being transmitted on those control channels. In another example, shown in FIG. 6B, the hub may include two transceivers 605a, 605b. In this example, one of the transceivers 605a may be dedicated to scanning the three control channels Cch1, Cch2 and Cch3 and may do so continuously, whilst at the same time the second transceiver is used to send and/or receive traffic in the data channel.

Returning back to FIG. 5, in step S502, the WBAN identifies those data channels on which the neighbouring WBANs are operating. In step S503, is determined whether or not there is present another WBAN that is operating on the same data channel as the WBAN in question.

In the present embodiment, steps S502 and S503 are carried out by constructing a table to record the control channel beacon information received from the WBANs in the vicinity. The columns of the table include, but are not limited to, Hub_ID, Data channel number and usage statistics. The entries in the table are obtained from listening to the control channel beacons being broadcast in the control channel(s) scanned by the hub. The Hub_ID and Data

Channel are obtained from the control channel beacons and the usage statistics are calculated based on activity of the neighbouring WBANs and their control channel beacons.

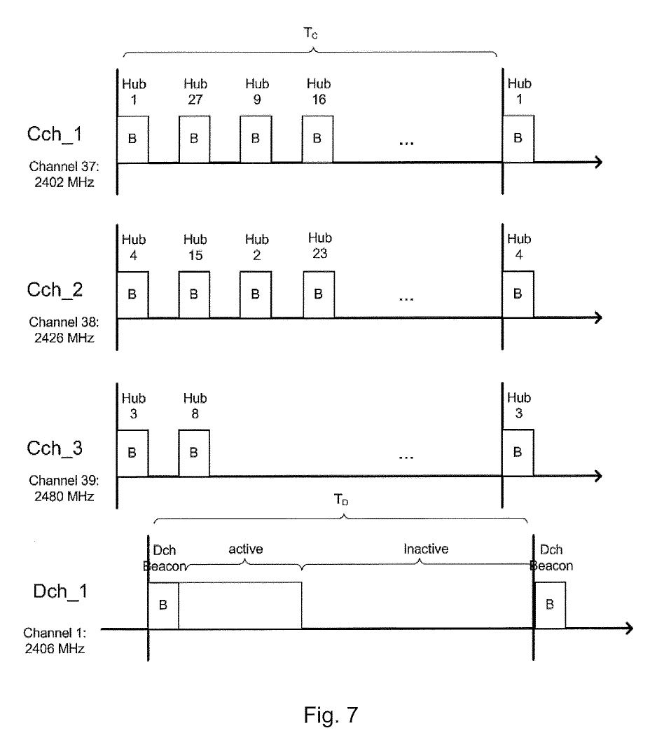

To provide an example of how the table may be constructed, reference is made to FIG. 7 which shows control channel beacons being transmitted by hubs of different WBANs at various intervals on three control channels Cch_1, Cch_2 and Cch_3. As explained above, each control channel beacon indicates the data channel on which the respective WBAN is currently operating. The three control channels are centred at 2042 MHz, 2426 MHz and 2480 MHz, respectively. Also shown in FIG. 7 is the activity in a data channel Dch_1 for the Hub 1.

Table 1 shows a table that may be constructed by Hub 1, based on the control channel beacons shown in FIG. 7.

TABLE 1
Sample construction of table based on control channel beacon information

| Control | | Data | |
|---|---|---|---|
| channels | Hub ID | channel | Usage Statistics |
| Cch_1 | Hub ID_1 | Dch = 1 | Since = 59 sec; [μ = 59 sec; σ = 0] |
| | Hub ID_27 | Dch = 32 | Since = 13 sec; [μ = 150 ms; |
| | | | σ = 2 sec] |
| | Hub ID_9 | Dch = 17 | Since = 5 sec; [μ = 3 sec; |
| | | | σ = 1 sec] |
| | Hub ID_16 | Dch = 26 | Since = 22 sec; [μ = 7 sec; |
| | | | σ = 2 sec] |
| | . . . | | |
| Cch_2 | Hub ID_4 | Dch = 3 | Since = 4 sec; [μ = 750 ms; |
| | | | σ = 500 ms] |
| | Hub ID_15 | Dch = 4 | Since = 26 sec; [μ = 26 sec; σ = 0] |
| | Hub ID_2 | Dch = 5 | Since = 58 sec; [μ = 10 sec; |
| | | | σ = 1 s] |
| | Hub ID_23 | Dch = 1 | Since = 4 msec; [μ = 0 ms; σ = 0] |
| | . . . | | |
| Cch_3 | Hub ID_3 | Dch = 13 | Since = 15 sec; [μ = 500 ms; |
| | | | σ = 500 ms] |
| | Hub ID_8 | Dch = 6 | Since = 34 sec; [μ = 34 sec; σ = 0] |
| | . . . | | |

The usage statistics shown in Table 1 indicate the activity status of the other WBANs in the vicinity, and help the hub maintaining the table to distinguish between hubs that are consistently active in a certain data channel, and those that are active more sporadically. The mean value (μ) indicates the average duration for which a hub has been active in a particular data channel. The variation (a) indicates the standard deviation in the duration of the hub being active in that data channel. The value “Since” refers to the length of time that has passed since the hub in question was first detected as being active in the vicinity.

In the event that the data channel column of the table includes an entry with the same data channel as that of the hub receiving the control channel beacon information, another WBAN with same data channel is said to have been detected. In the example shown above in Table 1,the control channel beacon of Hub 23, which is detected in the control channel Cch_2, indicates that the WBAN with Hub 23 is operating on the same data channel as that of Hub 1.

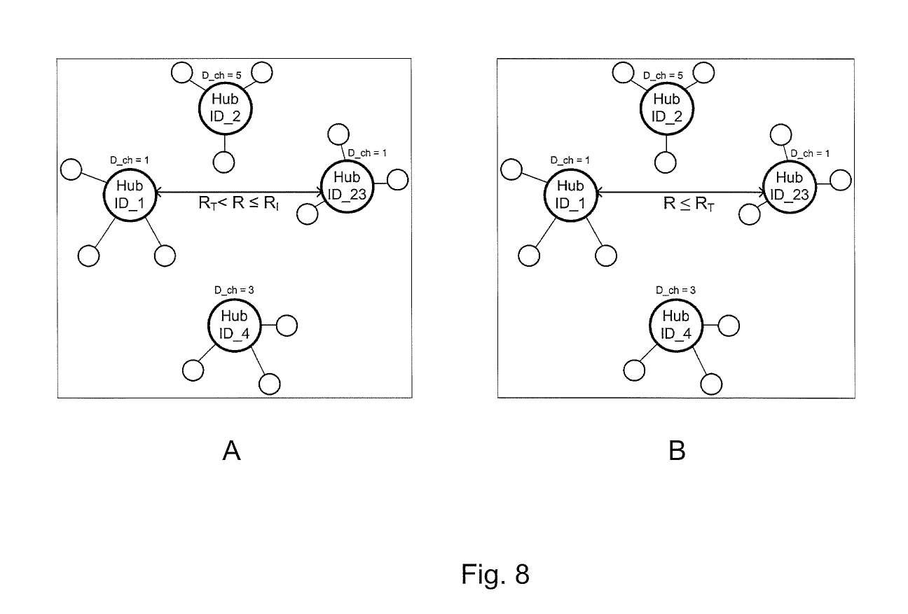

It will be understood that in order to detect the control channel information beacon broadcast by Hub ID_23, the distance R between Hub 1 (Hub ID_1) and Hub 23 (Hub ID_23) must be no greater than the transmission range R_{T} of Hub 23; this is explained with reference to FIGS. 8A and 8B. In FIG. 8A, the distance R between Hub 1 and Hub 23 is greater than R_{T}, hence Hub 1 will not detect the control channel beacons broadcast by Hub 23 and will remain ignorant of the fact that the WBAN to which Hub 23 belongs is operating on the same data channel (D_Ch 1) as Hub 1. In contrast, FIG. 8B shows the case in which the distance R between Hub 1 and Hub 23 is less than R_{T}, hence Hub 1 will be able to detect the control channel beacon sent from Hub 23 and determine that Hub 23 is operating on the same data channel.

Returning once more to FIG. 5, having determining that there is another WBAN in the vicinity that is operating on the same data channel, the WBAN takes action to try to mitigate interference between itself and the other WBAN (step S504). The WBAN may do so by adjusting its own communication parameters, by requesting that the other WBAN adjust its communication parameters, or by a combination of both.

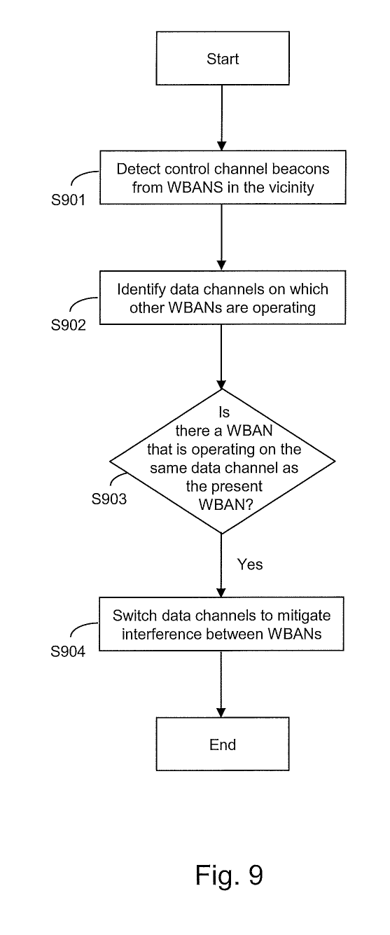

FIG. 9 shows an example in which the step of mitigating interference (step S904) is carried out by switching the data channel on which the WBAN is operating to a different data channel (it will be appreciated here that steps S901 to S903 reproduce steps S901 to S903 of FIG. 5, respectively). Although channel switching is a trivial solution to avoid interference, the minimal complexity that this technique involves makes it a suitable for consideration in embodiments described herein.

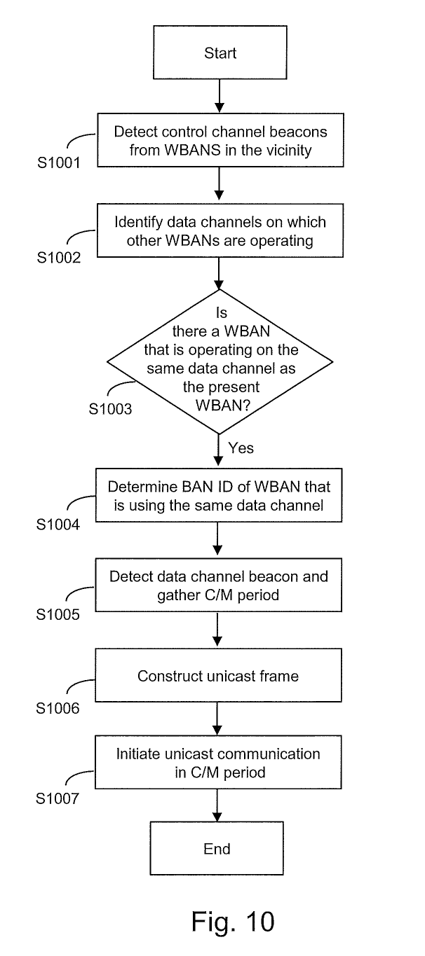

FIG. 10 shows an example of another embodiment in which the step of mitigating interference is carried out in a different way. As before, steps S1001 to S1003 are the same as steps S501 to S503 of FIG. 5.

Referring to step S1004 of the present embodiment, assuming that the hub has detected a second WBAN in the vicinity that is operating on the same data channel, the hub determines the ID of the hub in the second WBAN and initiates scanning to listen for the data channel beacon being broadcast by the second WBAN. The hub uses its inactive period (see FIG. 4) to scan for the data channel beacon.

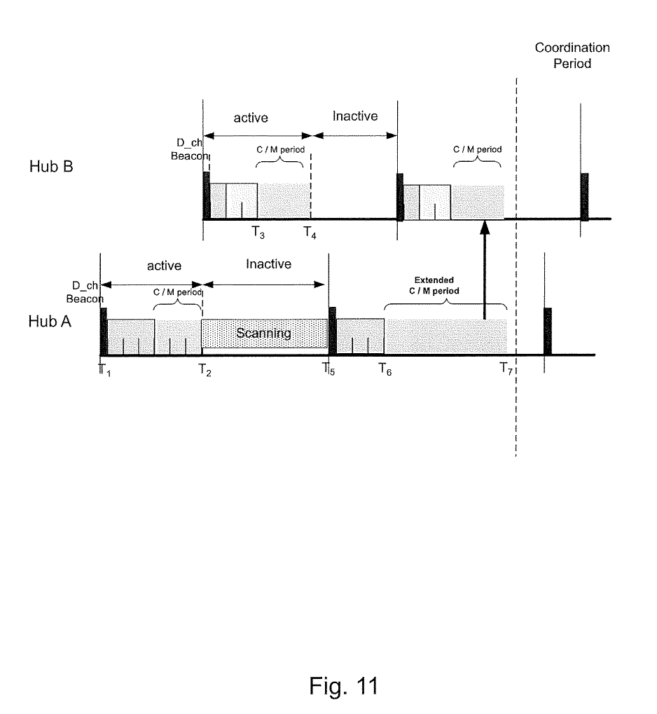

In step S1005, having detected the data channel beacon from the second WBAN, the hub determines the start and end of the Control and Management (C/M) period of the second WBAN. Following this, the hub extends its own C/M period into its (previously) inactive period, so as to overlap with that of the second WBAN. This process is illustrated in FIG. 11, which shows the data channel signalling for two hubs A and B which belong to different WBANs. Here, the Hub A has determined that Hub B is operating on the same data channel as itself, the Hub A having previously received a control channel beacon from Hub B specifying the data channel in question. At time T₁, Hub A broadcasts its data channel beacon and then enters its active period. At time T₂, Hub A ends its active period and enters its inactive period; Hub A now begins to scan the data channel for data beacons broadcast by Hub B and to determine the beginning and end points T₃, T₄ of Hub B's control/management period. At T₅, having determined those beginning and end points, Hub A broadcasts a new data channel beacon and enters a new active period, commencing its control/management period at T₆. Here, Hub A now extends the duration of its control/management period such that its control management period temporally overlaps with the control/management period of Hub B. In the present example, Hub A extends its control/management period to the time point T₇, at which point the control/management period of Hub B terminates; thus, the control/management periods of both Hub and Hub B end at the same time as one another.

In step S1006, having extended its own control/management period, the hub (Hub A) then constructs a frame that is unicast addressed to the hub of the second WBAN (Hub B). The frame is sent to the second WBAN to coincide with (i.e. during) the C/M period of the second WBAN (step S1007).

The unicast message may comprise a “Coordination Request” message, in which the hub notifies the second WBAN of the possibility of interference occurring between the two networks and requests that the second WBAN cooperate to mitigate interference, for example by one or other of them switching to another data channel and/or adjusting their active time frames in such a way that they do not coincide in time with one another. The pre-emptive communication may comprise a message packet with the sender's ID, the timing parameters of the sender and capabilities of the sender, etc. On receiving the coordination request, the second WBAN may in turn respond with a “Coordination Response” message; there may then follow a further exchange of messages in which the precise means for mitigating the interference (switching data channel, active period timing etc. are established).

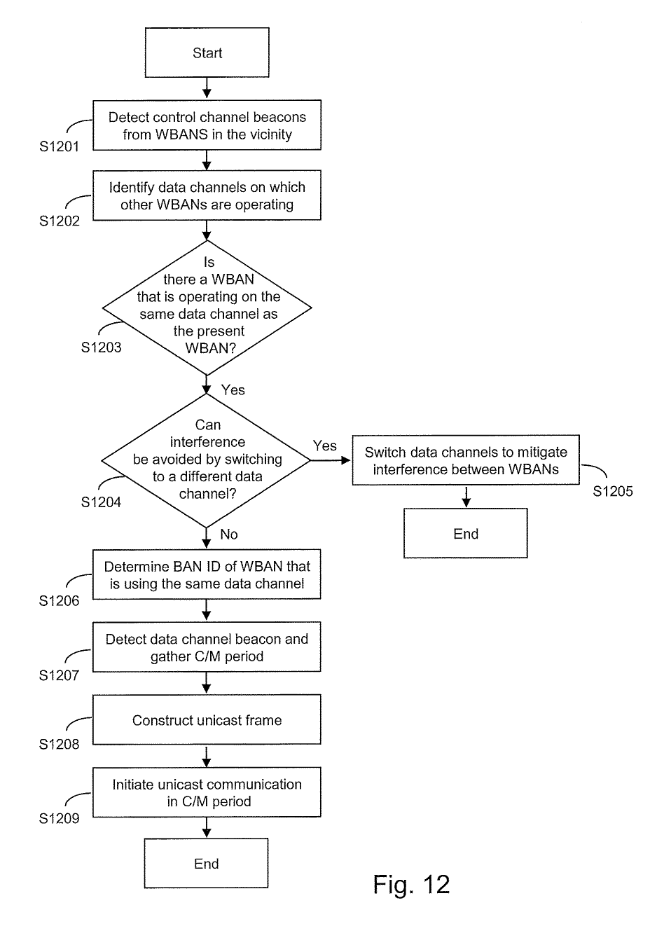

FIG. 12 shows an example of another embodiment, which combines features of the embodiments shown in FIGS. 9 to 11. As before, steps S1201 to S1203 reproduce steps S501 to S503 of FIG. 5. In step S1204, the hub determines whether it can avoid interference between itself and another WBAN that is operating on the same data channel by merely switching to another data channel. If so, the hub proceeds to switch data channels (step S1205); thus, the method proceeds in the same way as in step S904 of FIG. 9. In the event that it is determined that the hub cannot avoid interference merely by switching data channels (for example, because it is determined that there are other WBANs present in the vicinity of the hub that are operating on the other available data channels), the hub implement steps S1206 to S1209, which reproduce steps S1004 to S1007 of FIG. 10.

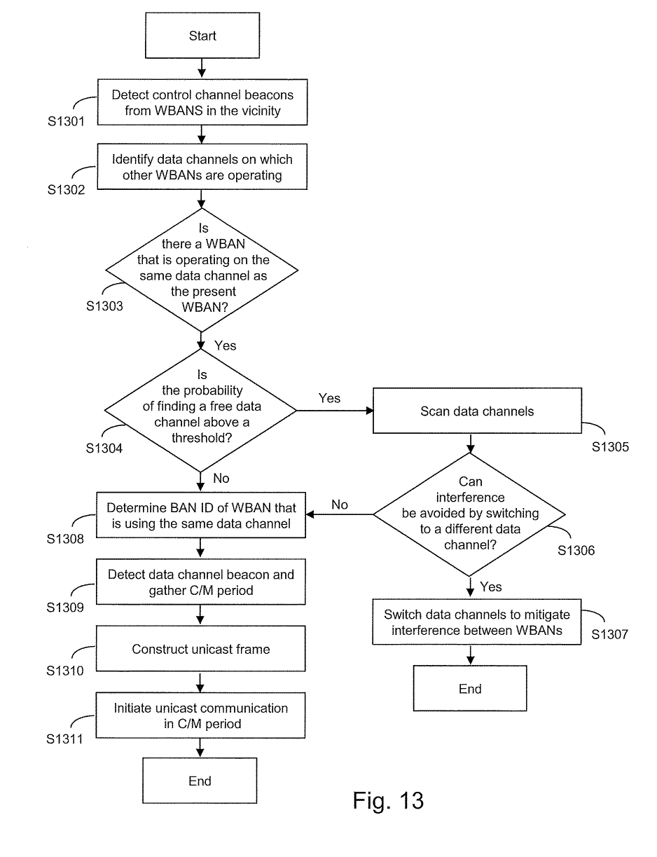

FIG. 13 shows an example in which the method of FIG. 12 is extended to include additional steps. As in FIG. 12, the hub determines whether there is a WBAN in the vicinity that is operating on the same data channel (step S1303). In step S1304, having determined there to be another WBAN operating on the same data channel, the hub evaluates whether or not the probability of finding a suitable alternative channel P [Finding a channel] is great enough to warrant scanning for a free data channel. In the event that steps S1301 and S1302 incorporate the construction of a table such as that shown by Table 1 above, step S1304 can be facilitated by considering the number of entries in that table. In particular, the density of WBANs in the vicinity and data channel occupancy from the table entries can provide an estimate of P. If P is large enough (for example, if P 0.5) then the hub will scan the data channels that are not listed in the table entries (step S1305). If a data channel is found that satisfies the application requirements for the WBAN (step S1306), then the hub will switch to the newly found data channel (step S1307). On the other hand, if a suitable data channel is not found, or the probability of finding such a channel is deemed to be too low in step S1304 to warrant scanning the data channels, then the method proceeds with steps S1308 to S1311, which reproduce the sequence of steps S1206 to S1209 in FIG. 12.

The embodiment shown in FIG. 13 can help reduce processing overhead by only requiring the hub to commence scanning of data channels in the event that it is considered likely that a free data channel will be found to be available.

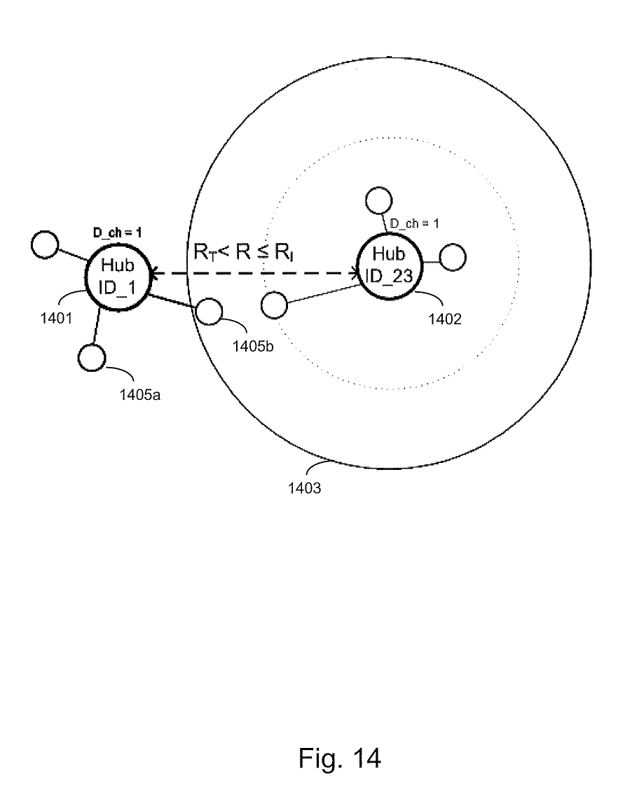

In the embodiments described above, the inter-WBAN detection and communication is carried out by the respective hubs of the WBANS. However, in some embodiments, the peripheral sensor nodes in the WBAN may serve to detect potentially interfering WBANs and notify their parent hub accordingly. An example of where this may be of value is shown in FIG. 14, in which there are two WBANs with respective hubs 1401, 1402 having identities Hub ID_1 and Hub ID_23. Both hubs are operating on the same data channel. The circle 1403 represents the edge of the transmission range of the second hub 1402. As can be seen, whilst the first hub 1401 is located outside the transmission range of the second hub 1402, one of the nodes 1405b that comprises part of the same WBAN as the first hub 1401 lies within range of the second hub 1402 and may be subject to interference from it.

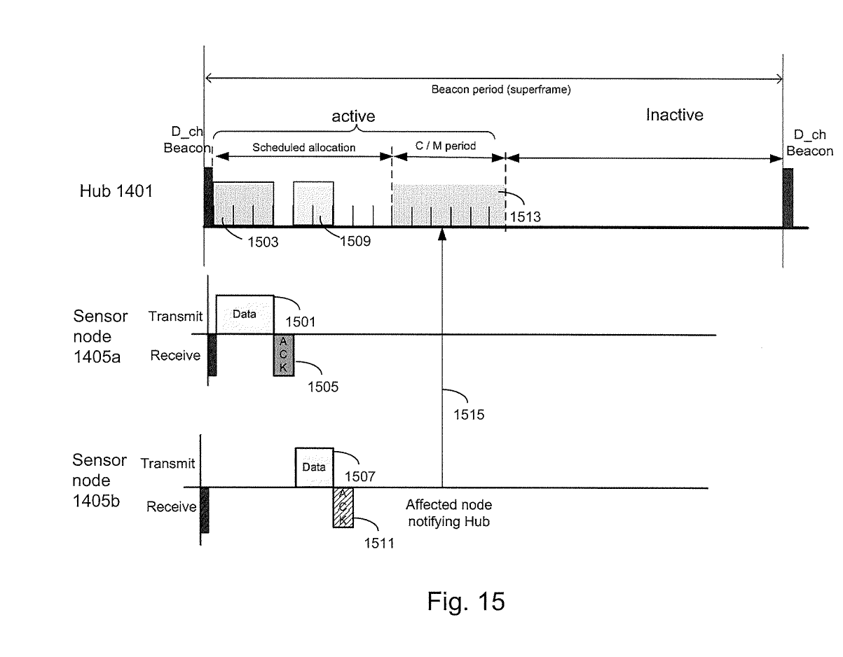

In the example shown in FIG. 14, since the hub 1401 itself lies outside the range of the neighbouring (interfering) hub 1402, the sensor node 1405 that is experiencing interference may transmit a signal to its parent hub 1401 to indicate the presence of the interfering WBAN. The sensor node 1405 may notify the hub 1401 during the hub's control/management period. FIG. 15 illustrates such a process: as shown in FIG. 15, the hub 1401 commences by transmitting a data channel beacon, which is received by the sensor nodes 1405a and 1405b belonging to the hub's network. The sensor node 1405a in turn transmits data 1501, which the hub 1401 listens out for in a first allocated period 1503 of its active period. The sensor node 1405a in turn receives an acknowledgement signal 1505 from the hub upon completion of its data transmission. After this, the second sensor node 1405b commences data transmission 1507, which the hub 1401 listens out for in a second allocated period 1509 of its active period. As before, the sensor node 1405b receives an acknowledgement signal 1511 from the hub 1401 upon completion of its data transmission. The second node 1405b is subject to interference from the second network with hub 1402 (see FIG. 14). Thus, as the hub 1401 enters its control/management period 1513, the second node 1405b transmits a notification signal 1515 to the parent hub, indicating the presence of the second hub 1402 in the vicinity, which is operating on the same data channel. The information contained in the notification 1515 from the sensor 1405b may then be used by the hub to populate a table such as that shown in Table 1.

Thus, in embodiments described herein, the information about other networks in the vicinity of a WBAN is available through control channel and/or data channel beacons and detection of those other networks is accomplished by exploiting information available through listening to those beacons.

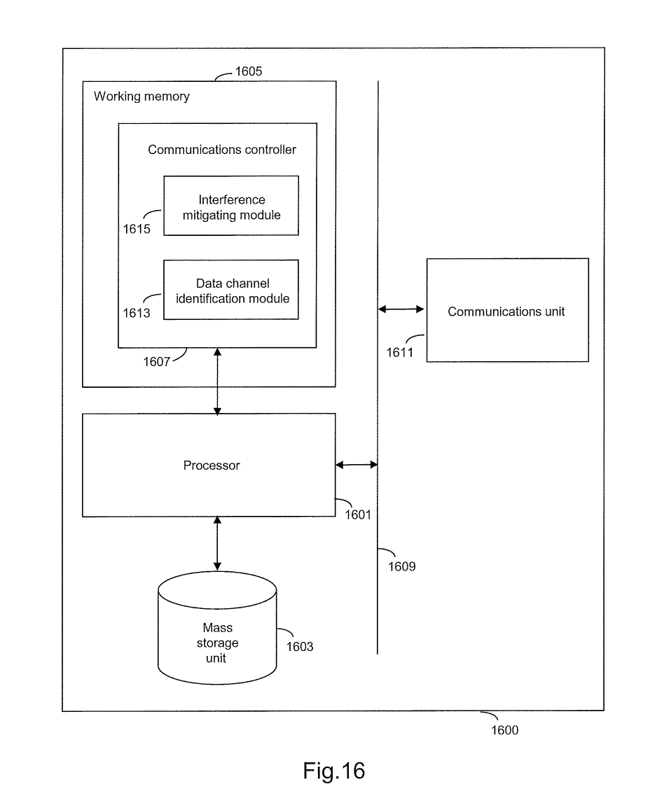

The reader will appreciate that the nodes of the WBANs described herein (including both the peripheral sensors and the network hubs) may be embodied as computing devices with means for wirelessly transmitting and/or receiving data from one another. An example of a typical computing device as used for a hub of such a WBAN is shown in FIG. 16, which provides means capable of putting an embodiment, as described herein, into effect. As illustrated, the computing device 1600 comprises a processor 1601 coupled to a mass storage unit 1603 and accessing a working memory 1605. As illustrated, a communications controller 1607 is represented as a software product stored in working memory 1605. However, it will be appreciated that elements of the communications controller 1607 may, for convenience, be stored in the mass storage unit 1603.

Usual procedures for the loading of software into memory and the storage of data in the mass storage unit 1603 apply. The processor 1601 also accesses, via bus 1609, a communications unit 1611 that operates to effect communications with the nodes in the WBAN, as well as for communicating with hubs of other WBANs in the vicinity (for example, where it is desired to send unicast messages to those other hubs, as described above). Typically, the communications unit 1611 will comprise one or more antennas to act as a transmitter and receiver for establishing a communications link with these other nodes.

The communications controller 1607 includes a data channel identification module 1613 and an interference mitigating module 1615. The data channel identification module 1613 is operable to identify the data channels on which other WBANs in the vicinity are operating, by analysing the data contained in the control channel beacons broadcast by those other WBANs. The interference mitigating module is in turn operable to determine whether one of the other WBANs is operating on the same data channel. In the event that the interference mitigating module determines that one of the other WBANS is operating on the same data channel, the interference mitigating module may select a new data channel on which the computing device is to operate. Alternatively, or in addition, the interference mitigating module may be operable to initiate unicast messaging to the neighbouring hub identified as operating on the same data channel by identifying the C/M period of neighbouring hub in a manner described above.

The communications controller software 1607 can be embedded in original equipment, or can be provided, as a whole or in part, after manufacture. For instance, the communications controller software 1607 can be introduced, as a whole, as a computer program product, which may be in the form of a download, or to be introduced via a computer program storage medium, such as an optical disk. Alternatively, modifications to an existing computing device 1600 can be made by an update, or plug-in, to provide features of the above described embodiment.

The system model and definitions of data/control channels and beacon frame formats contained herein are consistent with ETSI TC SmartBAN/SILMEE product development. Moreover, the embodiments described herein are applicable for any personal and body area networks. The feature of initiating communication unicastly addressed directly to a detected WBAN is particularly suitable for (albeit by no means limited to) use in intermittently active (low duty-cycle) networks.

While certain embodiments have been described, these embodiments have been presented by way of example only, and are not intended to limit the scope of the invention. Indeed, the novel methods, devices and systems described herein may be embodied in a variety of forms; furthermore, various omissions, substitutions and changes in the form of the methods and systems described herein may be made without departing from the spirit of the invention. The accompanying claims and their equivalents are intended to cover such forms or modifications as would fall within the scope and spirit of the invention.

1. A method of mitigating interference between two or more wide body area networks WBANs, the method comprising:
detecting, at a first WBAN, control channel beacons transmitted from one or more other WBANs, each control channel beacon specifying a data channel on which the respective WBAN is operating;
determining, based on the detected control channel beacons, whether one of the other WBANs is operating on the same data channel as the first WBAN and if so, adjusting one or more communication parameters of the first WBAN or requesting said one of the other WBANS to adjust its own communication parameters in order to mitigate interference between the first WBAN and said one of the other WBANs.

2. A method according to claim 1, wherein the first WBAN adjusts its one or more communication parameters by switching to operate on a different data channel.

3. A method according to claim 1, wherein the first WBAN adjusts its one or more communication parameters by adjusting the timing of an active period in the data channel on which it is operating.

4. A method according to claim 1, wherein the first WBAN maintains a record of WBAN activity in the vicinity, the record including a list of hubs from which control channel beacons have been detected and the data channels on which those hubs are operating, the record being updated on receipt of subsequent control channel beacons at the first WBAN.

5. A method according to claim 4, wherein the record of WBAN activity is used to generate statistics reflecting the duration for which each particular hub is active in a data channel, the statistics being updated on receipt of subsequent control channel beacons at the first WBAN.

6. A method according to claim 5, wherein the statistics include the mean duration for which each hub is active in a data channel and/or the standard deviation in the duration for which each hub is active in a data channel.

7. A method according to claim 4, wherein the record is stored in the form of a table.

8. A method according to claim 1, wherein the first WBAN notifies said one of the other WBANS of the possibility of interference between the first WBAN and said one of the other WBANs by:
determining a control/management period of said one of the other WBANs;
extending a control/management period of the first WBAN so as to coincide with the control/management period of said one of the other WBANs;
constructing a message for sending from the first WBAN to said one of the other WBANS; and
sending the message to said one of the other WBANs during the control/management period.

9. A method according to claim 1, wherein the first WBAN determines if the interference can be avoided by switching to operate a different data channel and if not, the first WBAN notifies said one of the other WBANS of the possibility of interference between the first WBAN and said one of the other WBANs by:
determining a control/management period of said one of the other WBANs;
extending a control/management period of the first WBAN so as to coincide with the control/management period of said one of the other WBANs;
constructing a message for sending from the first WBAN to said one of the other WBANS; and
sending the message to said one of the other WBANs during the control/management period.

10. A method according to claim 9 wherein the first WBAN scans a plurality of available data channels in order to determine whether the interference can be avoided by switching to a different data channel.

11. A method according to claim 10, wherein the step of scanning the plurality of channels is carried out subject to the probability of finding a free data channel being above a threshold.

12. A method according to claim 8 or 9, wherein the first WBAN uses the message to request the said one of the other WBANS to adjust its communication parameters in order to mitigate interference between the first WBAN and said one of the other WBANs.

13. A method according to claim 8 or 9, wherein the message is unicast addressed to said one of the other WBANS.

14. A method according to claim 1, wherein the first WBAN comprises one or more peripheral nodes and a hub,
wherein the one or more control channel beacons are detected at the peripheral node(s) and the information contained in the control channel beacons is relayed from the peripheral nodes to the hub, the hub determining whether to adjust one or more communication parameters of the WBAN on the basis of the received information.

15. A computer device for use as a node in a first wireless body area network WBAN, the computer device being configured to receive information contained in control channel beacons transmitted from one or more other WBANs;
the computer device comprising:
a data channel identification module for identifying, based on the received information, data channels on which the other WBANs are operating; and
an interference mitigating module configured to determine whether one of the other WBANs is operating on the same data channel as the first WBAN and if so, to adjust one or more communication parameters of the first WBAN or initiate a request for said one of the other WBANS to adjust its own communication parameters in order to mitigate interference between the first WBAN and said one of the other WBANs.

16. A non-transitory computer readable storage medium comprising computer executable instructions that when executed by a computer will cause the computer to carry out the method of claim 1.
